(19) United States
(12) Patent Application Publication
(10) Pub. No.: US 2006/0172316 A1
(43) Pub. Date: Aug. 3, 2006

(54) Quality value for nucleic acids

(76) Inventors: Paige Lynette Anderson, Redwood City, CA (US); Samar Jamol Lightfoot, San Francisco, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 11/048,152

(22) Filed: Jan. 31, 2005

(51) Int. Cl. C12Q 1/68; G06F 19/00; C12P 19/34

(52) U.S. Cl. 435/6; 435/91.2; 702/20

(57) ABSTRACT

A method of assessing the quality of an amplified nucleic acid sample is described. The method includes (a) separating the amplified nucleic acid sample to obtain a separation profile; (b) determining a first component of said separation profile corresponding to a first nucleic acid fraction; (c) determining a second component of said separation profile corresponding to a second nucleic acid fraction; and (d) assigning a quality value to the amplified nucleic acid sample based on the first component and the second component.

### FIELD OF THE INVENTION

The invention relates generally to methods of biochemical analysis. More specifically, the invention relates to analyzing a nucleic acid sample to determine quality of the sample.

### BACKGROUND OF THE INVENTION

Nucleic acid samples may be analyzed by a variety of techniques, including, for example, gel electrophoresis, HPLC, southern blotting, northern blotting, microarray hybridization experiments, sequencing techniques, and other techniques known in the art. The results of these techniques typically are dependent on the quality of the nucleic acids used.

A typical method of analyzing the quality of nucleic acids is gel electrophoresis. This usually involves a more or less subjective determination of the quality of the nucleic acids, such as visual inspection of gel electrophoresis results by an experienced researcher. Individual samples are graded for quality before being used in subsequent experiments and are rejected if they are not of suitable quality. This is a time-consuming and inefficient process, especially where many samples must be processed.

Nucleic acid amplification protocols are commonly used to produce quantities of nucleic acids for analysis and for other desired uses, e.g. genetic manipulation techniques. The nucleic acids to be amplified in the nucleic acid amplification protocols typically are isolated from a biological source (e.g. plant, animal, yeast, bacterium, virus) or are produced synthetically. The success of the amplification protocols can vary, based on the source of the initial nucleic acid and the quantity of the initial nucleic acid available to be amplified, as well as other parameters.

In addition, the ‘downstream’ (after amplification) techniques in which the amplified nucleic acid samples are used may be time-consuming and expensive to perform. Since the results of these ‘downstream’ techniques typically are dependent on the quality of the nucleic acids in the samples, it is desirable to have a method of determining the quality of the nucleic acids before performing the downstream techniques on the amplified nucleic acid samples.

Currently, lab-on-a-chip analyses, such as those available using the Agilent 2100 Bioanalyzer, provide accurate, reproducible, high resolution analyses of nucleic acid samples. These lab-on-a-chip analyses use microfluidic chips to analyze very small volumes of samples. In cases in which the samples are difficult or expensive to obtain, the small sample size required for analysis on microfluidic chips provides a practical solution to obtaining analysis of a nucleic acid sample. Results from lab-on-a-chip analyses provide a starting point to developing a measure of the quality of nucleic acid samples.

A method of determining quality of biomolecular samples is described in PCT publication No. WO2004/090780, which describes general methods of providing a measure of the quality of biological samples. Further references of interest include Guillaud-Bataille, M., et al., Nucleic Acids Res. 32(13): p. e112 (2004); Hughes, S., et al., Cytogenet Genome Res. 105(1): p. 18-24 (2004); Lage, J. M., et al., Whole Genome Research, 13(2): p. 294-307 (2003); Lavrrar, J. L. and P. J. Farnham, J Biol Chem. 279(44): p. 46343-49 (2004); Odom, D. T., et al., Science 303(5662): p. 1378-81 (2004); Kondo, Y., et al., Proc Natl Acad Sci USA, 101(19): p. 7398-403 (2004); Paez, J. G., et al., Nucleic Acids Res, 32(9): p. e71 (2004); Wang, G., et al., Genome Res, 14(11): p. 2357-66 (2004).

While advancements have been made in efficient analysis of nucleic acid samples, continuing improvements are needed.

### SUMMARY OF THE INVENTION

The invention addresses the aforementioned deficiencies in the art, and provides novel methods for assigning a measure of quality to a sample of nucleic acids. Thus, in certain embodiments, the present invention provides a method of assessing the quality of an amplified nucleic acid sample. The method includes (a) separating the amplified nucleic acid sample to obtain a separation profile; (b) determining a first component of said separation profile corresponding to a first nucleic acid fraction; (c) determining a second component of said separation profile corresponding to a second nucleic acid fraction; and (d) assigning a quality value to the amplified nucleic acid sample based on the first component and the second component.

Additional objects, advantages, and novel features of this invention shall be set forth in part in the descriptions and examples that follow and in part will become apparent to those skilled in the art upon examination of the following specifications or may be learned by the practice of the invention. The objects and advantages of the invention may be realized and attained by means of the instruments, combinations, compositions and methods particularly pointed out in the appended claims.

### BRIEF DESCRIPTION OF THE DRAWINGS

These and other features of the invention will be understood from the description of representative embodiments of the method herein and the disclosure of illustrative apparatus for carrying out the method, taken together with the Figures, wherein

FIG. 1 is an electropherogram showing results from Bioanalyzer analysis of a sample of an amplification reaction done without any genomic DNA input.

FIG. 2A is a histogram of results from an array hybridization experiment using the products obtained from a ‘zero genomic DNA input control’ of an amplification reaction.

FIG. 2B is a histogram of results from an array hybridization experiment using the products of an amplification reaction performed using 20 ng of genomic DNA.

FIG. 3 is an electropherogram showing results from Bioanalyzer analysis of a sample of the reaction products of an amplification reaction conducted with 50 ng of genomic DNA.

FIGS. 4A-4D are electropherograms showing results from Bioanalyzer analysis of samples of the reaction products of amplification reactions conducted with 50 ng (FIG. 4A), 5 ng (FIG. 4B), 0.05 ng (FIG. 4C), and 0 ng of genomic DNA.

FIGS. 5A and 5B are electropherograms showing results from Bioanalyzer analysis of sample of the reaction products of an amplification reaction conducted with 50 ng of genomic DNA (FIG. 5A) and with 50 ng of genomic DNA which has first been digested with AluI restriction enzyme (FIG. 5B).

To facilitate understanding, identical reference numerals have been used, where practical, to designate corresponding elements that are common to the Figures. Figure components are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible. However, the sequence described below is preferred.

It must be noted that, as used in the specification and the appended claims, the singular forms “a,” “an” and “the” include plural referents unless the context clearly dictates otherwise. Thus, for example, reference to “a peak” includes a plurality of peaks. Similarly, reference to “a nucleic acid” includes a plurality of nucleic acids.

Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation. In this specification and in the claims that follow, reference will be made to a number of terms that shall be defined to have the following meanings unless a contrary intention is apparent.

“Optional” or “optionally” means that the subsequently described circumstance may or may not occur, so that the description includes instances where the circumstance occurs and instances where it does not. For example, if a step of a process is optional, it means that the step may or may not be performed, and, thus, the description includes embodiments wherein the step is performed and embodiments wherein the step is not performed (i.e. it is omitted).

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. An “oligonucleotide” is a molecule containing from 2 to about 100 nucleotide subunits. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural (not usually occurring in nature) nucleotides, unnatural nucleotide mimetics such as 2′-modified nucleosides, peptide nucleic acids, oligomeric nucleoside phosphonates, and any polynucleotide that has added substituent groups, such as protecting groups or linking moieties. Polynucleotides are typically characterized by their length, e.g. in base pairs (bp) for double-stranded polynucleotides or in bases (b) for single-stranded polynucleotides.

“Moiety” and “group” are used to refer to a portion of a molecule, typically having a particular functional or structural feature, e.g. a linking group (a portion of a molecule connecting two other portions of the molecule), or an ethyl moiety (a portion of a molecule with a structure closely related to ethane). A moiety is generally bound to one or more other moieties to provide a molecular entity. As a simple example, a hydroxyl moiety bound to an ethyl moiety provides an ethanol molecule.

The term “array” encompasses the term “microarray” and refers to an ordered array of capture agents for binding to aqueous analytes and the like. An “array” includes any two-dimensional or substantially two-dimensional (as well as a three-dimensional) arrangement of spatially addressable regions (i.e., “features”) containing capture agents, particularly polynucleotides, and the like. A typical array may contain one or more, including more than ten, up to ten thousand features or more, e.g up to one hundred thousand features or more, in an area of less than 100 cm2, e.g., less than about 5 cm2, including less than about 1 mm2, or even smaller. Arrays can be fabricated by depositing (e.g., by contact- or jet-based methods) either precursor units (such as nucleotide or amino acid monomers) or pre-synthesized capture agent. An array is “addressable” when it has multiple regions of different moieties (e.g., different capture agent) such that a region (i.e., a “feature” or “spot” of the array) at a particular predetermined location (i.e., an “address”) on the array will detect a particular sequence. “Interrogating” the array refers to obtaining information from the array, especially information about analytes binding to the array. “Hybridization assay” references a process of contacting an array with a mobile phase containing analyte. An “array support” refers to an article that supports an addressable collection of capture agents.

“Isolated” or “purified” generally refers to isolation of a substance (compound, polynucleotide, protein, polypeptide, polypeptide, chromosome, etc.) such that the substance comprises a substantial portion of the sample in which it resides (excluding solvents), i.e. greater than the substance is typically found in its natural or un-isolated state. Typically, a substantial portion of the sample comprises at least about 5%, at least about 10%, at least about 20%, at least about 30%, at least about 50%, preferably at least about 80%, or more preferably at least about 90% of the sample (excluding solvents and buffer components (i.e. salts, buffer species, detergents, chelating agents) ) (where % is percent by weight). For example, a sample of isolated DNA will typically comprise at least about 5% total RNA, where percent is calculated in this context as mass (e.g. in micrograms) of total DNA in the sample divided by mass (e.g. in micrograms) of the sum of (total DNA+other constituents in the sample (excluding solvent) and buffer components (i.e. salts, buffer species, detergents, chelating agents)). Techniques for purifying polynucleotides of interest are well known in the art and include, for example, ion-exchange chromatography, affinity chromatography, precipitation, and sedimentation according to density. In typical embodiments, the sample is in isolated form prior to use in the present methods.

The term “sample” as used herein relates to a material or mixture of materials, typically, although not necessarily, in fluid form, containing one or more components of interest. The sample may be any nucleic acid sample, typically a sample containing nucleic acid (e.g. polynucleotides such as DNA or RNA) that has been isolated from a biological source, e.g. any plant, animal, yeast, bacterial, or viral source, or a non-biological source, e.g. chemically synthesized. The term “analyte” is used herein to refer to a known or unknown component of a sample. A “nucleic acid fraction” references a portion of a nucleic acid sample that has undergone a separation, and includes embodiments wherein the nucleic acid fraction is continuous and also embodiments wherein the nucleic acid fraction is discontinuous. In this regard, continuous means a single range of the separation profile (e.g. a single peak or multiple adjacent peaks including regions between the peaks) corresponds to the nucleic acid fraction. In this regard, discontinuous means more than one range of the separation profile (e.g. two peaks separated by an omitted portion of the separation profile or multiple peaks omitting the regions between the peaks) corresponds to the nucleic acid fraction.

Quality of a nucleic acid sample can be understood as a measure for its integrity. For example, a sample of nucleic acids will exhibit a high degree of integrity if the nucleic acids in the sample have survived extraction from cells or tissues without substantial degradation of the nucleic acids, e.g. without substantial breakage due to shearing forces or chemical breakdown of the nucleic acids. Quality is a measure of the intactness of the nucleic acids, and the suitability of the nucleic acids for the desired downstream technique. For samples of nucleic acids obtained via a process that includes an amplification reaction (e.g. an amplification reaction using phi29 DNA polymerase), quality can be expressed as a measure of the degree to which the product of the amplification reaction is representative of the starting material (e.g. the input genomic DNA). Typically, if the starting material is not of good quality the amplification product will not be of good quality, i.e. not representative of the starting material. “Quality value” references a measure of the quality of a nucleic acid sample. The quality value is obtained by analyzing a separation profile and selecting a plurality of components of the separation profile; a quality value based on the plurality of components is then assigned to the sample. For example, an algorithm or a mathematical relation may be applied to the plurality of components to provide a quality value. The algorithm may include scaling an unscaled score to provide a scaled quality score for the quality value. The algorithm or mathematical relation is selected to provide a meaningful measure of quality of the nucleic acid sample, particularly as it relates to a predetermined downstream process. The quality value typically provides a measure of the fitness of the amplified nucleic acid sample for use in a predetermined downstream process.

The term “predetermined” refers to an element that has a known characteristic prior to its use, e.g. whose identity is known prior to its use. For example, a “predetermined criterion” is a criterion that has a known characteristic prior to use of the predetermined criterion. In this context, an element may be known or characterized by name, sequence, molecular weight, its function, or any other attribute or identifier. In other word, “predetermined” references that the known characteristic has been ascertained or decided upon prior to engaging in the claimed method. “Desired”, as in “a desired product” or “desired separation method” generally means predetermined by the user based on experimental design considerations.

Thus, in certain embodiments, the present invention provides a method of assessing the quality of an amplified nucleic acid sample. The method includes (a) separating the amplified nucleic acid sample to obtain a separation profile; (b) determining a first component of said separation profile corresponding to a first nucleic acid fraction; (c) determining a second component of said separation profile corresponding to a second nucleic acid fraction; and (d) assigning a quality value to the amplified nucleic acid sample based on the first component and the second component.

In typical embodiments, separating the amplified nucleic acid sample includes taking an aliquot of the amplified nucleic acid sample and subjecting the aliquot to a separation method to obtain the separation profile. A separation profile is a collection of data obtained by observing the separation of the amplified nucleic acid sample. The separation profile may be obtained using any separation method known in the art, e.g. electrophoresis, chromatography, capillary electrophoresis, or other known methods. “Observing” in this context references obtaining data by measuring a signal resulting from the separation of the amplified nucleic acid sample, e.g. from an observable moiety in the nucleic acid sample, such as a fluorophore, a chromophore, a property of the nucleic acid itself, an interaction of the nucleic acid with part of the detection system, or any other method of measuring the result of a separation known in the art. The separation profile may be raw data or may be processed, e.g. normalized, smoothed, scaled, integrated, related to a standard and/or control, having artifacts removed, subtracting baseline, or other methods of processing data as are known in the art or apparent from the description herein. The data may be in any format, e.g. tabular, graphical, entries in an electronic datafile stored in computer memory or on a recording medium, or any other format. Once the data is obtained, it may be recorded or displayed. Recording the data and/or displaying the data may facilitate determining components of the separation profile. The data typically can be represented by a trace of observed signal (which is a function of the quantity of nucleic acid) versus a variable such as time, mobility, retention coefficient, size, length, molecular weight, or the like (depending on the separation method). For example, the separation profile may be represented by a chromatogram or an electropherogram. The separation profile typically may include peaks in the data. Such peaks typically indicate a population of nucleic acids that have similar characteristics and/or behave similarly under the separation conditions employed.

A component is a portion of the data from the separation profile. A component may include data from one or more portions from the separation profile, e.g. elution peaks or electropherogram peaks, or other selected data, e.g. selected to cover a given time period or selected relative to a control or standard. As used herein, “correspond,” “corresponding” or other like language, references the relationship between a portion of data from the separation profile and the separated portion of nucleic acid that is observed to result in that portion of the data. For example, a portion of data is said to correspond to the separated portion of nucleic acid that is observed to give that portion of data. Similarly, a separated portion of nucleic acid is said to correspond to the portion of data that results from observing that separated portion of nucleic acid.

“Determining” a component references selecting data from one or more portions from the separation profile to define that component. For example, a component (e.g. a first component or a second component) may be selected which includes one or more peaks, or other selected data, e.g. selected to cover a given time period or selected relative to a control or standard. In certain embodiments, the selection may be based on one or more predetermined criteria selected from molecular weight, length of nucleic acid, the relative position of a standard or control, time period, retention coefficient, the magnitude of the data, absolute or relative mobility, the presence of a label in the portion of nucleic acid corresponding to a portion of the separation profile, or any predetermined criteria equivalent to these, or any other predetermined criteria, or a combination thereof. In certain embodiments, the predetermined criteria provides a threshold value, e.g. data from one or more portions of the separation profile that are less than the threshold value are included in the first component and data from one or more portions of the separation profile that exceed the threshold value are included in the second component. In some embodiments, the predetermined criteria provide a range of values of the predetermined criteria, and the first component includes selected data that lie within a first given range of values of the predetermined criteria, and the second component includes selected data that lie within a second given range of values of the predetermined criteria. As used herein, “selecting” or “selection,” or like language, when used in the context of selecting data or a portion of a separation profile includes both selecting data to be included in a component and omitting the data (or portion of the separation profile) not selected to be included in the component.

In particular embodiments, the predetermined criteria is length of the nucleic acids, i.e. data for a component are selected based on the length of nucleic acids in the separated portion of nucleic acids that corresponds to the data. In some such embodiments, a first component is selected to include a portion of the separation profile corresponding to nucleic acids that are greater than a threshold length; also, a second component is selected to include a portion of the separation profile corresponding to nucleic acids that are less than the threshold length. In certain embodiments a first component is selected to include a portion of the separation profile corresponding to nucleic acids having a length in a first given range (e.g. a peak in the separation profile); also, a second component is selected to include a portion of the separation profile corresponding to nucleic acids length in a second given range (e.g. a different peak in the separation profile).

In some embodiments, the threshold length is in the range from about 200 base pairs (bp) to about 8000 bp, e.g in the range from about 500 bp to about 7500 bp, typically in the range from about 1000 bp to about 7000 bp, more typically in the range from about 1500 to about 6500 bp, e.g. in the range from about 2000 to about 6000 bp. In some embodiments, the threshold length is in the range from about 2500 bp to about 6000 bp, e.g. in the range from about 3000 bp to about 5000 bp, typically in the range from about 3500 bp to about 4500 bp. In particular embodiments, the threshold length is about 4000 bp (plus or minus about 200 bp). In typical embodiments, the length of a selected portion of nucleic acids corresponding to a portion of the separation profile may be determined by conducting a separation (under the same conditions) on a “ladder sample” comprising nucleic acids having several different defined lengths, and then comparing mobilities of the components of the ladder sample with the mobility of the selected portion of nucleic acids in question.

In particular embodiments, the predetermined criteria is chosen from length, size, molecular weight, or mobility of the DNA, as provided in the separation profile. In embodiments in which the predetermined criteria include size or molecular weight for determining the first component and second component, the size or molecular weight typically will be the approximate (e.g. within about 10%) size or molecular weight equivalent of DNA having the length set forth elsewhere herein e.g. the previous paragraph. For example, assuming about 660 dalton per bp, a threshold length in the range from about 200 to about 8000 bp would give an approximate equivalent threshold molecular weight in the range from about 132 kdal to about 5280 kdal.

In certain embodiments the predetermined criteria, e.g. the threshold length, are determined empirically. For example, a series of amplification reactions is performed on varying amounts of an initial sample of nucleic acids. Each member of the series has a different amount of the initial sample, e.g. amplification reactions are performed using 0.01 nanograms, 0.1 nanograms, 1 nanogram, 10 nanograms, and 100 nanograms of nucleic acid from the initial sample, as well as an amplification reaction in which no (0 nanograms) nucleic acid from the sample is included. The solutions of amplified nucleic acids resulting from the amplification reactions are then subjected to a separation method to obtain separation profiles for each of the solutions of amplified nucleic acids. In some embodiments, the separation profiles may then be compared with respect to the amount of initial sample, and portions of the separation profiles that correlate to the amount of initial sample may be selected for the first component. Also in such embodiments, portions of the separation profiles that do not correlate to the quantity of initial sample may be selected for the second component. In other embodiments portions of the separation profiles that correlate to the amount of a desired product may be selected for the first component, and portions of the separation profiles that do not correlate to the desired product may be selected for the second component. In this way, by using varying amounts of the initial sample in the amplification reactions and analyzing the data from the separation profiles, criteria may be chosen which provide for selecting data from one or more portions from the separation profile to define the first component and second component. These criteria may serve as the predetermined criteria for methods of assessing the quality of nucleic acids in a sample as described herein. In particular embodiments, these predetermined criteria provide for selection of a range of data for the first component and a range of data for the second component, and the first component and second component are used to assign a quality value to the sample. In other embodiments, the threshold length may be determined by experiment, using results obtained from a selected downstream process to iteratively determine the threshold that gives acceptable results in the downstream process.

Typically, the values of the predetermined criteria for selecting the first component and second component (e.g. the threshold length, or a first given range for selecting the first component and a second given range for selecting the second component) are selected with regard to factors including the amplified DNA sample (e.g. source, method of amplification), the desired separation method (e.g. the Bioanalyzer analysis method, conditions of the separation method), and a predetermined downstream process. If one of these factors changes, the values of the predetermined criteria (e.g. the threshold length) may need to be re-established to befit the factors. As an example, if the polymerase enzyme used to provide the amplified DNA sample is changed, the threshold length may change. For example, in embodiments in which the amplification reaction includes phi29 DNA polymerase, the threshold length is typically in the range from about 2500 bp to about 6000 bp. However, the threshold length may be lower if the amplified DNA sample was prepared using a polymerase enzyme having lower processivity than phi29, e.g. the threshold length may be in the range from about 1000 to about 3000 bp, or even in the range from about 500 bp to about 1500 bp.

As another example, change in the elution conditions or other experimental parameter of the separation method may result in the separation profile being altered, in which case the predetermined criteria may need to be re-established. For example, if a component is determined based on migration time, the value of the criteria may need to be re-established to reflect a different mobility under the changed elution conditions. Other things that may affect threshold besides enzyme include reaction conditions such as buffer components (salts, metals, etc.), reaction temperature, reaction time, and the like.

As used herein, a predetermined downstream process is any process for which an amplified nucleic acid sample (e.g. an amplified DNA sample) is expected to be used, for example an analytical method employing the amplified nucleic acid sample. Typical predetermined downstream processes include (but are not limited to): analysis by microarray, preparative scale methods, recombinant DNA methods, or any other methods in which the quality and/or quantity of the amplified nucleic acids may affect the outcome of the predetermined downstream process. Typical downstream processes also include (but are not limited to): chromosome painting, Southern blotting, restriction length polymorphism analysis, genotyping of single nucleotide polymorphisms, subcloning, DNA sequencing, and aCGH (array-based comparative genomic hybridization) analysis.

Typically, the method of the present invention is used to assign a quality value to an amplified nucleic acid sample to determine whether to proceed with the predetermined downstream process. The predetermined downstream process may be difficult, time-consuming, and/or expensive to perform, thus it is desirable to provide a quality value to indicate the quality of the amplified nucleic acid sample prior to proceeding with the predetermined downstream process so that amplified nucleic acid samples with unsatisfactory quality values may be excluded from (are not subjected to) the predetermined downstream process. It should be noted that an amplified nucleic acid sample may have an acceptable quality value for one predetermined downstream process but the same sample with the same quality value may be unacceptable for a different predetermined downstream process.

In particular embodiments, the separation profile of an amplified nucleic acid sample includes two major peaks. In such embodiments, a threshold value intermediate the two peaks is selected to serve as a predetermined criterion for determining the first component and second components of separation profiles of subsequently obtained amplified nucleic acid samples. The first component may be selected such that it includes one of the two major peaks, and the second component may be selected such that it includes the other of the two major peaks. In typical embodiments, the “two major peaks” are characterized in that they are the two largest peaks of the separation profile (as measure by area under the curve) and that the sum of the areas under the curves of the two major peaks comprises at least about 40% (e.g. at least about 50%, typically at least about 60%, more typically at least about 70%, still more typically at least about 80%) of the area under the curve (when such standard practice as baseline subtraction is employed).

In an embodiment of the invention, the quality value is ascertained automatically in conjunction with a nucleic acid analysis system that includes a microfluidic separation device which provides the separation of the amplified nucleic acid sample. The nucleic acid analysis system receives data resulting from the separation of the nucleic acid sample, obtains a separation profile, automatically determines at least a first component and second component of the separation profile, automatically applies an algorithm or mathematical relation to the at least first and second components to provide a quality value, and automatically assigns the quality value to the nucleic acid sample. The automatic functions described may be facilitated by the use of appropriate software. The software may include functions for receiving separation profile data, selecting peaks from the separation profile, determining at least a first component and second component of the separation profile, and assigning a quality value based on the first component and the second component. In an embodiment, the software includes a function to allow a user to manually adjust and/or select portions of the separation profile (e.g. peaks); the software also includes a function which calculates the quality value based on the user input and the separation profile. In particular embodiments, the software includes functions to compute the peaks automatically, to determine the first component and second component, and to assign quality value.

In certain embodiments, the quality value is calculated using the first component and second component. For example, the quality value may be obtained by calculating a quantity (denoted R1) (e.g. area under curve, quantity of nucleic acid, or other quantity based on the first component) corresponding to the first component, calculating a quantity (denoted R2) (e.g. area under curve, quantity of nucleic acid, or other quantity based on the second component) corresponding to the second component (R2), and calculating the quality number based on R1 and R2. In certain embodiments, the quality value is calculated using a ratio based on the first component and the second component. Such a ratio may be selected from, e.g., R1/R2, R2/R1, R1/(R1+R2), R2/(R1+R2), or other such formula. In particular embodiments, the quality number is calculated using a scaling function that assigns a quality number based on R1 and R2.

Nucleic acid samples are typically obtained from a biological source, e.g. from microorganisms (e.g., viruses, bacteria and fungi), animals (e.g., cows, pigs, horses, sheep, dogs and cats), hominids (e.g., humans, chimpanzees, and monkeys) and plants. The samples can come from tissues or tissue homogenates or fluids of an organism and cells or cell cultures. Thus, for example, samples can be obtained from whole blood, serum, semen, saliva, tears, urine, fecal material, sweat, buccal tissue, skin, spinal fluid, tissue biopsy or necropsy, and hair. Samples can also be derived from ex vivo cell cultures, including the growth medium, recombinant cells and cell components. In comparative studies (e.g. to identify potential drug or drug targets), one sample can be obtained from diseased cells and another sample from non-diseased cells, for example. Nucleic acids may be isolated from such sources using protocols that are well known in the art. Such isolated nucleic acids may then be amplified to yield the amplified nucleic acid sample.

To obtain an amplified nucleic acid sample, an initial specimen containing nucleic acid (e.g. DNA) to be amplified is contacted with a polymerase enzyme under conditions sufficient to result in amplification of nucleic acid in the initial specimen to yield the amplified nucleic acid sample. In particular embodiments, the polymerase enzyme may be, e.g. phi29 DNA polymerase or Bst DNA polymerase. In specific embodiments, the amplified DNA sample is obtained using a phi29 DNA polymerase enzyme, such as that available from QIAGEN. Protocols for amplification are well known in the art and are typically available from commercial sources of the polymerase enzymes and also from references in the literature. The amplified nucleic acid sample may be obtained using any nucleic acid amplification protocol, such as MDA (multiple displacement amplification), DOP-PCR (random or degenerate oligonucleotide-primed PCR), RCA (Rolling Circle Amplification), RCA-RCA (Restriction and Circularization-Aided Rolling Circle Amplification); PCR based amplification of fragmented DNA using linkers.

The separation profile may be obtained using any separation method known in the art, e.g. electrophoresis, chromatography, capillary electrophoresis. In certain embodiments the separation method may separate the nucleic acids based on molecular weight, length, charge, or other characteristic of the nucleic acid. In this regard, “based on size” (or molecular weight) does not require that the selection be based solely on the size (or molecular weight) of the nucleic acids.

In particular embodiments, separating the amplified nucleic acid sample comprises using a microfluidic separation device adapted to performing nucleic acid separations. As used herein, a microfluidic separation device is one that uses less than about 50 ul applied sample and less than about 10 ml of reagent solutions/buffers to provide results in typical use of the device. In some embodiments, a microfluidic device is used for performing biochemical separations on small amounts (e.g. <50 microliter, typically <20 microliter, more typically <10 ul, still more typically <5 ul, or even <2 ul, e.g. 1 ul or less) of applied sample. Concentration of DNA used in such samples is generally in the range from about 0.1 ng/ul to about 10,000 ng/ul, typically from about 1 ng/ul to about 2500 ng/ul, more typically from about 5 ng/ul to about 1000 ng/ul, still more typically from about 10 ng/ul to about 500 ng/ul, yet more typically from about 20 ng/ul to about 300 ng/ul. In particular embodiments, the method of the present invention includes separating an amplified DNA sample using a microfluidic separation device adapted to separating the amplified DNA sample on the basis of size to provide a separation profile. Agilent Technologies, Inc. (Palo Alto, Calif.) supplies kits for performing microfluidic separations of biological samples known as LabChip™ kits. These kits include microfluidic separation devices for use in compatible bioanalyzer apparatus, such as the Agilent 2100 bioanalyzer. Other microfluidic separation systems adapted to separation of nucleic acids samples may be employed.

### EXAMPLES

The practice of the present invention will employ, unless otherwise indicated, conventional techniques of synthetic organic chemistry, biochemistry, molecular biology, and the like, which are within the skill of the art. Such techniques are explained fully in the literature, such as in Sambrook et al. Molecular cloning, 2nd Ed., Cold Spring Harbor Laboratory Press (1989).

The following examples are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric. Standard temperature and pressure are defined as 20° C. and 1 atmosphere.

Experimental

Amplified DNA samples were obtained using an amplification protocol which included contacting isolated genomic DNA with phi29 DNA polymerase (available from QIAGEN) under conditions sufficient to result in amplification of the genomic DNA. Standard amplification protocols were used, such as are known in the art. The amplified DNA samples were then heat denatured and ˜200 ng of each amplified DNA sample were loaded onto a Bioanalyzer Lab-on-a-chip device and analyzed. The equipment used was an Agilent 2100 Bioanalyzer with RNA 6000 Nano LabChip kits (Agilent Technologies, Inc., Palo Alto, Calif.). Separation profiles were obtained for each of the amplified DNA samples and were displayed as plots (electropherograms) of observed signal (related to quantity of nucleic acid) versus migration time.

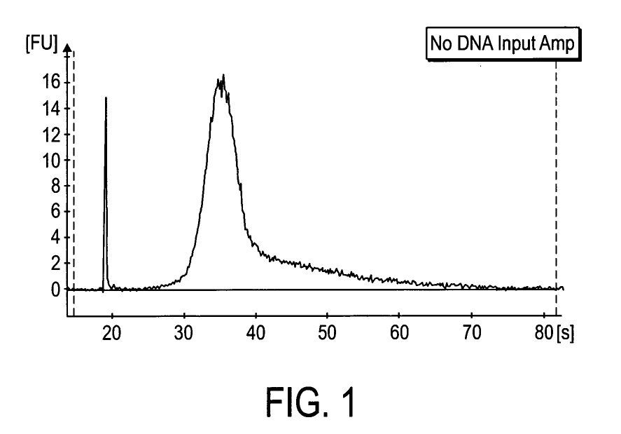

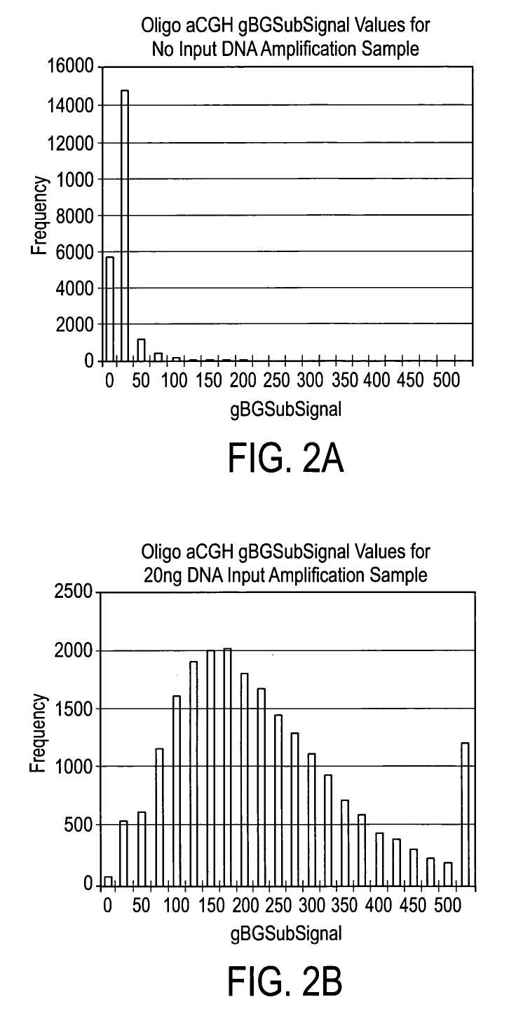

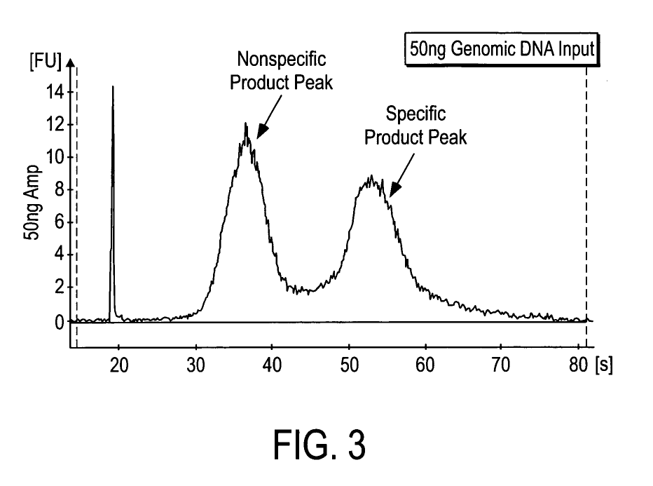

The amplification reaction can result in the formation of a population of DNA products that appear to be artifacts of the amplification reaction. Bioanalyzer analyses of amplification reactions done without any genomic DNA template input into the amplification reaction show a resulting electropherogram peak migrating out in the 30-40 second range, FIG. 1. To study the products of this ‘zero input genomic DNA control’ of the amplification reaction, a sample of the products was applied to a custom array designated WGA-alphaV2 as well as a commercial catalog array designated human genome CGH microarray 44A (Agilent Technologies, Inc.) under conditions allowing for binding of DNA to the arrays. These products did not hybridize to the experimental probes (capture agents) on the arrays, resulting in an absence of hybridization signal. FIG. 2A shows results from such an array hybridization experiment, showing that the background subtracted signals obtained from reading the features of the array were quite low. In contrast, when genomic DNA (20 ng) was included in the amplification reaction, the products of the reaction mixture did show significant binding to the array. FIG. 2B shows results from such an array hybridization experiment, showing that the signals obtained from reading the features of the array indicate significant binding of the amplified genomic DNA to the array. FIG. 2A and FIG. 2B are histogram plots, which graphically illustrate a large data set that has been parsed into bins or discreet groupings represented on the X-axis. These bins are selected in a way that provides for meaningful representation of the data. In FIGS. 2A and 2B the bins were set in increments of 25 (0, 25, 50, 75, 100 . . . to 500). So the first column of data, the 0 bin on the x-axis, shows all the features on the array that have background subtracted signals (BGSubSignals)<0. The second column, bin 25, shows the features with BGSubSignals>=0 but <25. The third column, bin 50, shows the features with BGSubSignals>=25 but <50, and so on. The y-axis shows how many of the features on the array fall into each bin. For instance, in FIG. 2A in bin 25 (>=25 but <50) there are about 550 features and bin 50 has slightly more. The last bin is called the ‘more’ bin. Any features with BGSubSignals>=500, or greater than the last bin the user specifies, are put in this bin. From FIGS. 2A and 2B it is obvious that products of amplification reactions performed using 0 ng (No Input DNA, FIG. 2A) and 50 ng of input DNA (FIG. 2B) give very different array hybridization results. The 50 ng amplification reaction product gives an expected signal distribution for this array platform with a median BGSubSingal value of ˜200. However, the No Input DNA amplification reaction product has the vast majority of features with BGSubSignals<50 and many are <0, which means that most signals on the features are at background levels. From the No Input DNA hybridization none of the data is useful—it is biologically meaningless. (Note that only data for the biological probes (capture agents) on the array are shown. All positive and negative control probes were removed from the data set.)

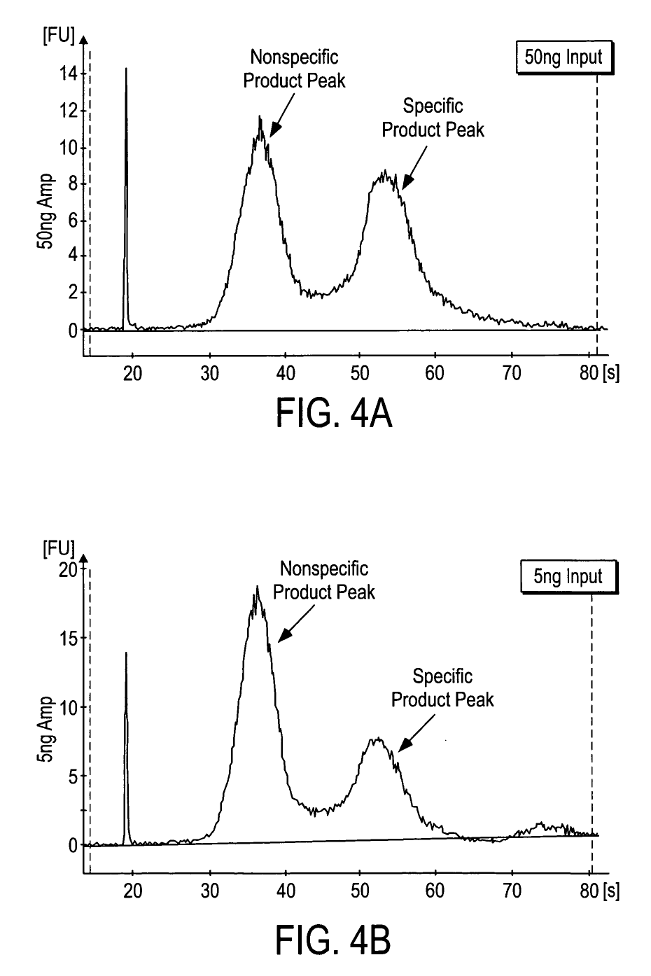

For an amplification reaction with a standard input of genomic DNA there were two distinct peaks in the plots (electropherograms). A first peak migrated out in the ˜30-40 second range, followed by a second peak which migrated out in the ˜50-60 second range. The first peak was ascertained to represent DNA fragments that were smaller in size (shorter than ˜4000 bp) than the DNA fragments that were represented by a second peak (longer than ˜4000 bp). FIG. 3 is an electropherogram showing results from Bioanalyzer analysis of a sample of the reaction products of an amplification reaction conducted with 50 ng of genomic DNA. FIG. 3 shows the first peak migrating out at ˜30-40 seconds and the second peak migrating out at ˜50-60 seconds. The second peak represents amplified genomic DNA fragments (>4000 bp).

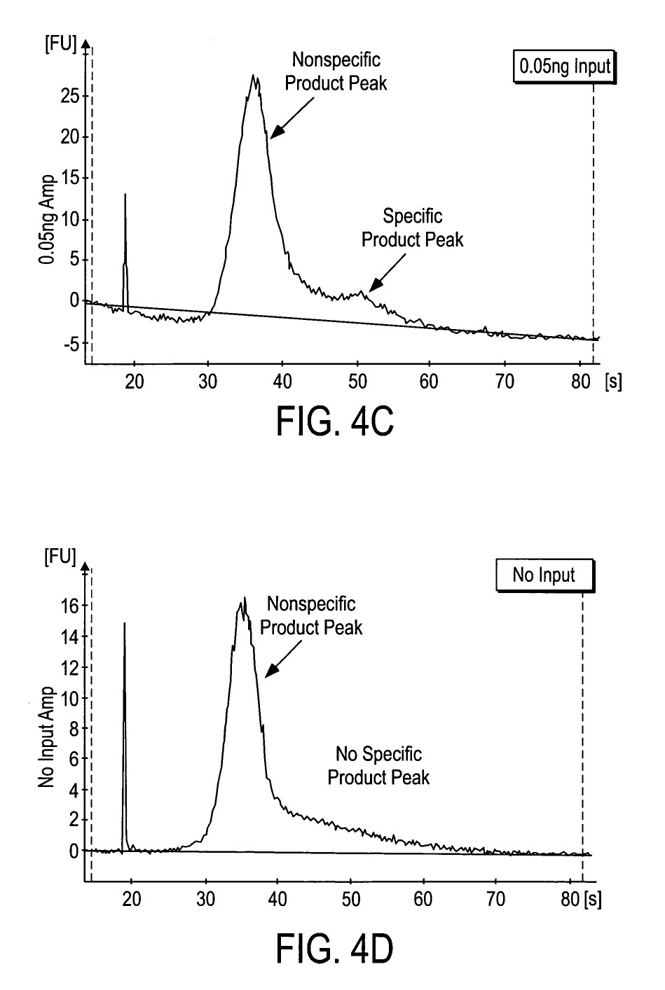

Further experiments using decreasing amounts of genomic DNA in the amplification reaction were performed. FIGS. 4A-4D are the electropherograms showing results from Bioanalyzer analysis of samples of the reaction products of amplification reactions conducted with 50 ng (FIG. 4A), 5 ng (FIG. 4B), 0.05 ng (FIG. 4C), and 0 ng of genomic DNA. These results show that as the amount of input DNA into an amplification reaction is decreased, the first nonspecific peak increases in size and the second specific peak decreases in size.

As an example of the present method, the peak migrating out at ˜50-60 seconds that represents amplified genomic DNA longer than ˜4000 bp was selected to be the first component of the separation profile. Also, the peak migrating out at ˜30-40 seconds that represents DNA fragments smaller than ˜4000 bp was selected to be the second component of the separation profile. The ratio of these peaks can be used to calculate how much of the amplified material needs to be used in the downstream application(s), or if the ratio is too low it may be concluded that it is best not to proceed with the current amplified sample and to repeat the amplification. For example, if the ratio of the first component to the second component is 1.0 this means that 50% of the sample is the DNA fragments smaller than ˜4000 bp and 50% is the amplified genomic DNA longer than ˜4000 bp. This information may be used in downstream processes as follows: If 6 μg of DNA is required for a labeling reaction but if only 50% of the amplified DNA represents amplified genomic DNA (3 μg) then the amount to be added to the labeling reaction should be increased to 12 μg to make the amplified genomic DNA in the reaction 6 μg.

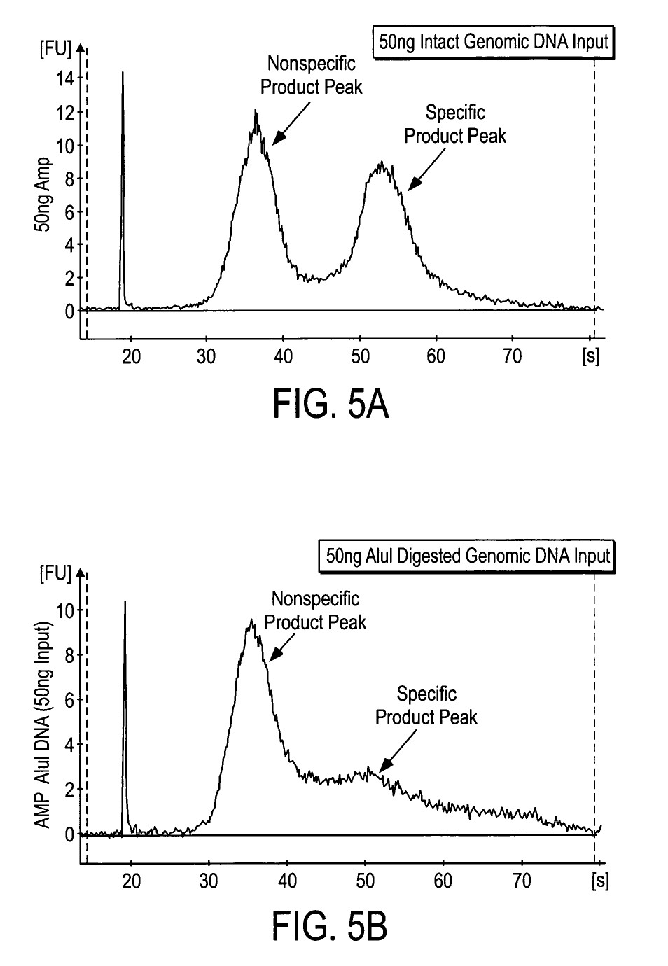

We next investigated the effect of degraded DNA on the amplification reaction. Genomic DNA was digested with AluI restriction enzyme to mimic the random degradation seen in some genomic DNA samples, e.g. FFPE extracted DNA. The AluI digested genomic DNA was used as the amplification template in an amplification reaction using phi29 DNA polymerase, and very little or no discernable products migrated out in the ˜50-60 second peak. FIGS. 5A and 5B illustrate these results. FIG. 5A is an electropherogram showing results from Bioanalyzer analysis of a sample of the reaction products of an amplification reaction conducted with 50 ng of genomnic DNA. FIG. 5B is an electropherogram showing results from Bioanalyzer analysis of a sample of the reaction products of an amplification reaction conducted with 50 ng of genomic DNA which has first been digested with AluI restriction enzyme. FIGS. 5A and 5B illustrate that degraded DNA is a poor template for amplification with phi29 DNA polymerase (or under the selected conditions).

It should be noted that, although we observed two peaks eluting at ˜30-40 seconds and at ˜50-60 seconds, the method of the present invention does not rely on absolute migration time, but instead involves determination of a first component and a second component of a separation profile and assigning a quality value to the amplified DNA sample, as described above. In the experiments described above, the quality value can be considered to be a ratio of the second component to the first component, but any other method of deriving a quality value based on the first component and the second component may be used.

It is now apparent that a quality value can be assigned to an amplified nucleic acid sample, wherein the quality value may be useful in downstream processes performed using the amplified nucleic acid sample, e.g. to adjust the amount of sample used. The quality value may also be used to determine whether the downstream analysis should be performed on the sample, thereby possibly avoiding expensive and time-consuming effort on downstream processing using low-quality samples. The quality value thus provides a useful improvement in the art.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties, provided that if there is any conflict in definitions or interpretation of claims, the terms and definitions set forth herein shall control.

What is claimed is:

1. A method of assessing the quality of an amplified nucleic acid sample, the method comprising:
(a) separating the amplified nucleic acid sample to obtain a separation profile;
(b) determining a first component of said separation profile corresponding to a first nucleic acid fraction;
(c) determining a second component of said separation profile corresponding to a second nucleic acid fraction; and
(d) assigning a quality value to the amplified nucleic acid sample based on the first component and the second component.

2. The method of claim 1 wherein determining the first component comprises selecting a portion of the separation profile corresponding to nucleic acids having a length greater than a threshold value, and wherein determining the second component comprises selecting a portion of the separation profile corresponding to nucleic acids having a length less than the threshold value.

3. The method of claim 1, wherein the threshold length is in the range from about 200 bp to about 8000 bp.

4. The method of claim 1, wherein the amplified nucleic acid sample is obtained using an amplification protocol using phi29 DNA polymerase or Bst DNA polymerase.

5. The method of claim 1, wherein the threshold length is in the range from about 2000 bp to about 6000 bp and the amplified nucleic acid sample is obtained using an amplification protocol using phi29 DNA polymerase.

6. The method of claim 1, wherein separating the amplified nucleic acid sample comprises using a microfluidic separation device adapted to performing nucleic acid separations.

7. The method of claim 1, wherein the amplified nucleic acid sample is subjected to a downstream process if the quality value is within an acceptable range.

8. The method of claim 1, wherein determining the first component comprises selecting a portion of the separation profile based on a predetermined criterion selected from molecular weight, length of nucleic acid, relative position of a standard or control, time period, retention coefficient, absolute or relative mobility, the presence of a label in the portion of nucleic acid corresponding to the portion of the separation profile, magnitude of data, or a combination thereof, and wherein determining the second component comprises selecting a portion of the separation profile based on the predetermined criterion.

9. The method of claim 1, wherein assigning the quality value comprises calculating the quality value based on the amount of nucleic acid corresponding to the first component and the amount of nucleic acid corresponding to the second component.

10. The method of claim 1, wherein assigning the quality value comprises deriving a quantity corresponding to the first component (R1), deriving a quantity corresponding to the second component (R2), and calculating the quality value based on R1 and R2.

11. The method of claim 1, wherein assigning the quality value comprises deriving a quantity corresponding to the first component (R1), deriving a quantity corresponding to the second component (R2), and calculating the quality value using a scaling function that assigns a quality number based on R1 and R2.

12. The method of claim 1, wherein the separation profile comprises a plurality of peaks and the first component comprises at least one peak of the plurality of peaks.

13. The method of claim 12, wherein the first component comprises at least two peaks of the plurality of peaks.

14. The method of claim 12, wherein the second component comprises at least one peak of the plurality of peaks.

15. The method of claim 14, wherein the second component comprises at least two peaks of the plurality of peaks.
